(19) United States
(12) Patent Application Publication
(10) Pub. No.: US 2020/0179696 A1
(43) Pub. Date: Jun. 11, 2020

(54) SUPPRESSING SEIZURES WITH LOW FREQUENCY STIMULATION OF THE CORPUS CALLOSUM

(71) Applicant: Case Western Reserve University, Cleveland, OH (US)

(72) Inventors: Dominique M. Durand, Solon, OH (US); Nicholas H. Couturier, Cleveland, OH (US)

(21) Appl. No.: 16/682,698

(22) Filed: Nov. 13, 2019

Related U.S. Application Data
(60) Provisional application No. 62/775,980, filed on Dec. 6, 2018.

(51) Int. Cl. A61N 1/36

(52) U.S. Cl. CPC A61N 1/36064; A61N 1/36157; A61N 1/36153; A61N 1/36175

(57) ABSTRACT

Cortical seizures can be suppressed with low frequency stimulation of the corpus callosum. An electrical stimulation signal with a frequency of less than 20 Hz can be applied to the corpus callosum in a patient's brain for a time. Hyper-excitability (indicative of seizure activity) of a target neural tissue within a cortex of the patient's brain that is activated by the corpus callosum can be suppressed. In fact, the hyper-excitability is reduced based on the stimulation.

### RELATED APPLICATIONS

This application claims the benefit of U.S. Provisional Application Ser. No. 62/775,980, filed Dec. 6, 2018, entitled “CORPUS CALLOSUM LOW-FREQUENCY STIMULATION SUPPRESSES SEIZURES”. The entirety of this provisional application is hereby incorporated by reference for all purposes.

### GOVERNMENT SUPPORT

This invention was made with U.S. government support under 2R01 NS060757 05A1 and 5T32EB004314-20 awarded by the National Institutes of Health (NIH). The government has certain rights in this invention.

### TECHNICAL FIELD

The present disclosure relates generally to seizure suppression and, more specifically, to systems and methods to suppress seizures with low frequency stimulation of the corpus callosum.

### BACKGROUND

Epilepsy is one of the most prevalent neurological disorders, affecting approximately 1% of the world's population. Each year, it is estimated that there are between 16 and 51 new patients diagnosed with an epilepsy disorder per 100,000 people. Unfortunately, around 20% of these patients do not respond to antiepileptic medication. The most common alternative to medication is surgical resection; however most patients are not eligible for resection due to an inability to identify a well-defined epileptogenic region that is not colocalized with any eloquent cortex. Even of the patients eligible for surgery, only a little more than half experience freedom from seizures.

Brain stimulation technologies have been developed around the unmet need for seizure suppression in patients for whom medication and surgery are not viable options. Electrical stimulation has the advantage of being less invasive, adjustable, and reversible compared to surgical resection. Recently, two deep brain stimulation techniques have been granted FDA approval for use in treating refractory epilepsies. Although many grey matter stimulation targets have been investigated along with different stimulation parameters including frequency only the responsive neurostimulation (RNS) system and stimulation of the anterior nucleus of the thalamus (ANT) have been implemented clinically. Both of these techniques rely on electrical stimulation of grey matter targets with frequencies >100 Hz. Stimulation of white matter tracts at low frequency (<100 Hz, such as between 1 and 20 Hz, for example) is an attractive alternative to grey matter stimulation. One study related to stimulation of the fornix at low frequency to reduce seizures in humans with intractable epilepsy. Stimulating the corpus callosum at low frequency may similarly reduce cortical seizures.

### SUMMARY

The present disclosure relates to systems and methods to suppress seizures with low frequency stimulation of the corpus callosum. The seizures can be, for example, cortical seizures, hippocampal seizures, or the like.

In an aspect, the present disclosure can include a system that can suppress seizures with low frequency stimulation of the corpus callosum. The system includes a stimulation generator that is configured to generate an electrical stimulation signal with a frequency of less than 50 Hz. The system also includes at least one electrode configured to apply the electrical stimulation signal to a corpus callosum in a patient's brain for a time. Hyper-excitability of a target neural tissue within a cortex of the patient's brain that is activated by the corpus callosum is reduced based on the stimulation.

In another aspect, the present disclosure can include a method for suppressing seizures with low frequency stimulation of the corpus callosum. An electrical stimulation signal with a frequency of less than 50 Hz can be applied to the corpus callosum in a patient's brain for a time. Hyper-excitability (indicative of potential seizure activity) of a target neural tissue within a cortex of the patient's brain that is activated by the corpus callosum can be suppressed. The hyper-excitability can also be reduced for period of time following the removal of the stimulation.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic diagram showing an example of a system that can suppress seizures with low frequency stimulation of the corpus callosum in accordance with an aspect of the present disclosure;

FIG. 2 is a top view diagram of an example brain with hemispheres and the corpus callosum (CC);

FIG. 3 is a top view diagram of the example brain of FIG. 2 with electrodes of the system of FIG. 1 implanted;

FIG. 4 is a process flow diagram illustrating a method for suppressing seizures with low frequency stimulation of the corpus callosum according to another aspect of the present disclosure;

FIG. 5 is a process flow diagram illustrating a method for configuring an electrical signal for stimulation of the corpus callosum based on feedback control according to yet another aspect of the present disclosure;

FIG. 6 shows the placement of electrodes and microsyringes within the brain of an animal;

FIG. 7 shows the characterization of an acute focal cortical model of epilepsy;

FIG. 8 shows the corpus callosum (CC) stimulation frequency range that produced an inhibitory effect on cortical seizures;

FIG. 9 shows the CC stimulation frequency range that produced an inhibitory effect on hippocampal seizures;

FIG. 10 shows the spatial extent of CC stimulation;

FIG. 11 shows the effect of CC stimulation on a different epileptic focus; and

FIG. 12 shows the effect of different stimulations on focal areas of seizures.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “corpus callosum” can refer to a wide, thick nerve tract, including a flat bundle of commissural fibers (including thousands of axons), located beneath the cerebral cortex in the brain. The corpus callosum connects the left and right sides of the brain, allowing for communication between both hemispheres.

As is used herein, the terms “cerebral cortex” and “cortex” can refer to the outer layer of neural tissue of the cerebrum of the brain that is separated into two cortices by the longitudinal fissure that divides the cerebrum into the left and right hemispheres. The two hemispheres are joined beneath the cortex by the corpus callosum.

As used herein, the term “hippocampus” can refer to a brain structure embedded deep in the temporal lobe of each cerebral cortex.

As used herein, the term “stimulation” can refer to delivery of a signal (e.g., an electrical signal) to activate conduction within a nerve or group of nerves.

As used herein, the term “high frequency” can refer to a frequency greater than 100 Hz.

As used herein, the term “low frequency” can refer to a frequency less than 100 Hz. For example, a low frequency can be less than 50 Hz. As another example, a low frequency can be less than 15 Hz. As a further example, a low frequency can be less than 10 Hz. As another example, a low frequency can be less than 5 Hz.

As used herein, the term “seizure” can refer to unusual and/or uncontrolled electrical activity in a patient's brain. A seizure can include physical effects, such as abnormal movement or behavior.

As used herein, the term “cortical seizure” can refer to a seizure with a focal area within the cerebral cortex.

As used herein, the term “hippocampal seizure” can refer to a seizure with a focal area within the hippocampus.

As used herein, the term “hyper excitability” can refer to the unusual or uncontrolled electrical activity in a patient's brain characteristic of a seizure. The term hyper excitability also refers to the state if being easily involved in hyperexcitable activity

As used herein, the term “suppress” can mean remove something. When something is suppressed, 50% or more, 55% or more, 60% or more, 65% or more, 70% or more, 75% or more, 80% or more, 85% or more, 90% or more, 95% or more, 97% or more, 98% or more, 99% or more, or 100% of something is removed (e.g., when seizures are suppressed, more than 50% of the seizures suffered by a patient can be stopped or eliminated).

As used herein, the term “proximal” can refer to being near (not in direct contact) or in direct contact.

As used herein, the term “patient” can refer to a mammal suffering from a cortical seizure disorder.

### II. Overview

Stimulating the corpus callosum at low frequency for a time reduces the incidence of seizures (e.g., cortical seizures, hippocampal seizures, and the like). The seizures can be reduced for the time and may even be reduced for a period extending beyond application of stimulation to the corpus callosum. Although the functional characteristics of callosal fibers have been the subject of active debate, the corpus callosum is thought to consist predominantly of excitatory axons that synapse on both pyramidal cells and inhibitory neurons in the corresponding cortical hemispheres. Regardless of the exact nature of the transcallosal fibers, stimulation of the corpus callosum has been shown to suppress seizures similarly to the effect observed in the hippocampus with commissural stimulation. Indeed, by stimulating specific tracts of the corpus callosum, seizures can be inhibited in foci located in the cortical regions innervated by fibers of the corpus callosum.

### III. Systems

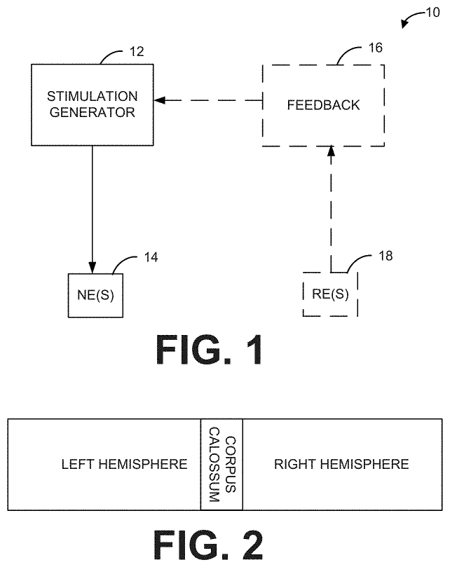

An aspect of the present disclosure can include a system 10 (FIG. 1) that can suppress seizures with low frequency stimulation (also referred to as LFS) of the corpus callosum. The corpus callosum can be stimulated for a time (e.g., according to a treatment plan including a treatment time customized for the patient and the type of seizure; as a non-limiting example, the treatment time can include a series of on and off times (e.g., on 30 seconds, off 2 minutes) for a treatment time (e.g., 1 hour)). The suppression of the seizures (and/or corresponding reduction in hyper-activity) can occur (1) as the stimulation is delivered and (2) may extend for a period after the time (after the stimulation of the corpus callosum is complete). For example, the period after the time can extend for at least two hours longer than the time. As another example, the period after the time can extend for at least four hours longer than the time. As a further example, the period after the time can extend for at least one day longer than the time. As yet another example, the period after the time can extend for at least seven days longer than the time.

The low frequency stimulation can include electrical stimulation signals with a frequency less than 100 Hz. For example, a low frequency can be less than 50 Hz. In another example, a low frequency can be less than 20 Hz. As another example, a low frequency can be less than 15 Hz. As a further example, a low frequency can be less than 10 Hz. As another example, a low frequency can be less than 5 Hz. Low frequency stimulation is different than traditional deep brain stimulation, which includes frequencies greater than 100 Hz.

In some instances, the stimulation of the corpus callosum at one or more specific points in the corpus callosum can reduce hyper-excitability characteristic of seizure activity in at least one target area. The target area can be an area of the brain remote from the corpus callosum, such as the cerebral cortex, the hippocampus, or the like. The seizures that can be suppressed by stimulating the corpus callosum include cortical seizures, hippocampal seizures, and the like. Cortical seizures can be dispersed throughout the cerebral cortex and/or include one or more focal areas within the cortex. Hippocampal seizures can be dispersed throughout the hippocampus and/or include one or more focal areas within the hippocampus. As shown in FIG. 2, the corpus callosum spans between the two hemispheres of the brain with fibers of the corpus callosum invading both hemispheres. Although the functional characteristics of callosal fibers have been the subject of active debate, the corpus callosum is thought to consist predominantly of excitatory axons that synapse on both pyramidal cells and inhibitory neurons in the corresponding cortical hemispheres. Regardless of the exact nature of the transcallosal fibers, stimulation of the corpus callosum has been shown to suppress seizures similarly to the effect observed in the hippocampus with commissural stimulation (a different area of the brain, the frontal dorsal commissure (FDC)).

The system 10 includes one or more stimulation generators 12 configured to generate the electrical waveforms with low frequency used to stimulate the corpus callosum (the electrical waveforms may include one or more parameters, such as a type of waveform, a pulse width, a current intensity, a current amplitude, and a voltage amplitude, or the like that can be configured by the one or more stimulation generators 12) and one or more electrodes (NEs) 14 to apply the electrical waveform to the corpus callosum. The one or more stimulation generators 12 can be external to the patient's body, implanted within the patient's body, and/or a portion being within the patient's body and a patient external to the patient's body. Similarly, the one or more electrodes (NEs) 14 can be implanted within the patient's brain (e.g., depth electrodes) and/or external to the patient's brain (e.g., surface electrodes). Using multiple electrodes (NEs) 14 can create a greater size of tissue affected by the stimulation of the corpus callosum.

In some instances, the electrical waveforms configured by the one or more stimulation generators 12 may be voltage waveforms. In other instances, the electrical waveforms configured by the one or more stimulation generators 12 may be current waveforms. In either instance, one or more additional circuit elements may be present before the electrical waveform is delivered to respective electrodes (NEs) 14 (e.g., amplifiers, filters, convertors (I to V or V to I), etc.).

Additionally, the system can include one or more recording electrodes (RE(S)) 18, a feedback unit 16, and, in some instances, additional circuitry (e.g., an amplifier, a filter, or the like). The feedback unit 16 can be a feedback circuit that is coupled to or part of the stimulation generator 12 to receive a feedback signal from the at least one recording electrode (RE(S)) 18. The RE(S) 18 can be placed at one or more positions that can provide information related to the area of the brain affected by the stimulation of the corpus callosum (e.g., information indicative of conduction within the target area to indicate the hyper-excitability). The information can be processed by the feedback unit 16 and sent to the one or more stimulation generators 12, which can change one or more parameters of the stimulation based on the information.

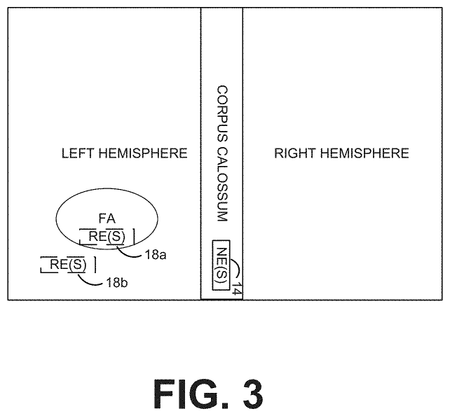

For example, as shown in FIG. 3, one or more electrodes (NEs) 14 can be proximal to an area of the corpus callosum (specifically chosen based on the area of the cortex targeted). A focal area of a seizure within a hemisphere of the cortex is represented by a circle. The position that the stimulation is delivered (and electrodes are oriented and positioned) within the corpus callosum is designed to affect only the target focal area of the seizure and not the other parts of the cortex. This can minimize side effects associated with the stimulation. Recording electrodes 18a, 18b can be placed at one or more positions within (18a) and/or outside (18b) the focal area. Information about the focal area related to the hyper-excitability, such as information related to conduction, can be recorded by one or more of the recording electrodes 18a, 18b and sent to the feedback unit 16, which can process the information and send the processed information to the one or more stimulation generators 12, which can adjust one or more parameters of the electrical signal based on the processed information. In other instances, the recording electrode 18a placed within the focus can be used to make sure the electrode (NE(S)) 14 is in the correct place, stimulating the correct axons. The feedback unit 16 in such instances can provide an output related to whether the electrode (NE(S)) 14 is in the correct place (and/or whether the correct axons are activated in the corpus callosum) and/or can provide an autonomous movement of the electrode (NE(S)) 14 into a more proper position.

### IV. Methods

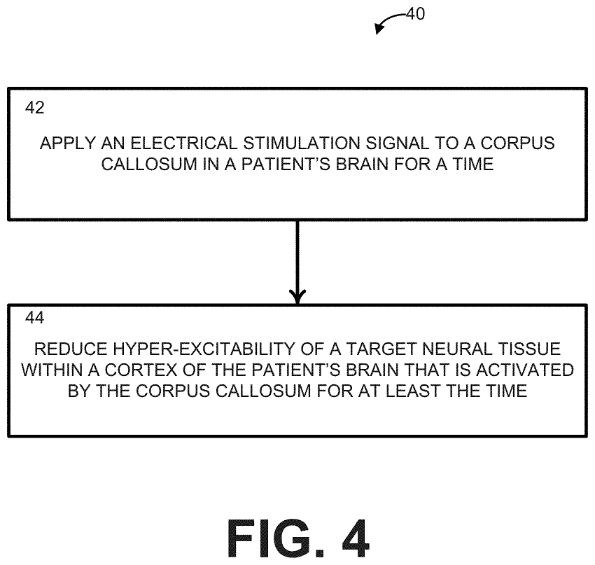

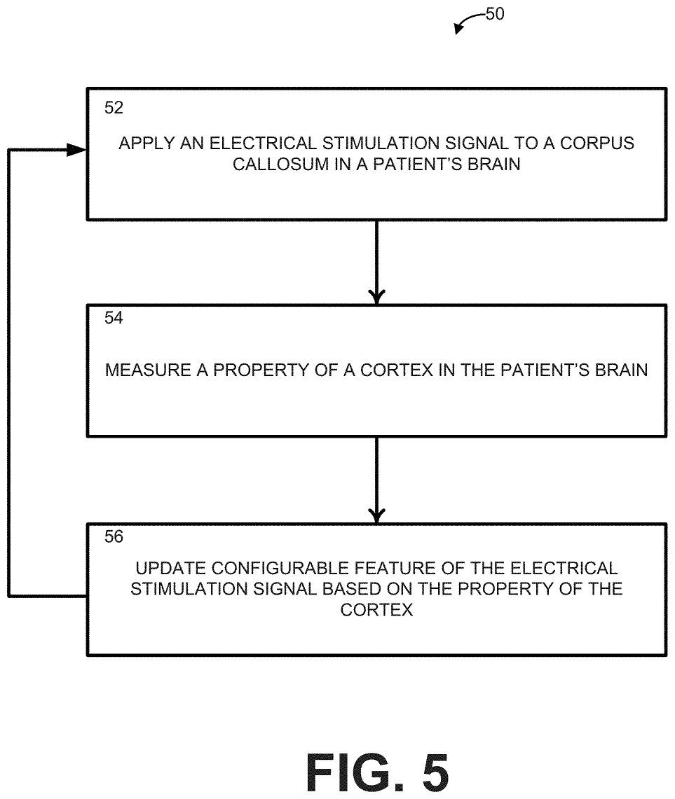

Another aspect of the present disclosure can include a method 40 for suppressing seizures with low frequency stimulation of the corpus callosum, as shown in FIG. 4. Moreover, a further aspect of the present disclosure can include a method 50 for configuring an electrical signal for stimulation of the corpus callosum based on feedback control, as shown in FIG. 5. The methods 40 and 50 can be executed using the system 10 shown in FIG. 1, for example.

For purposes of simplicity, the methods 40 and 50 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 40 and 50.

Referring now to FIG. 4, illustrated is a method 40 for suppressing seizures with low frequency stimulation of the corpus callosum. At Step 42, an electrical stimulation signal with a frequency of less than 50 Hz can be applied to a corpus callosum in a patient's brain for a time. As a further example, a low frequency can be less than 20 Hz. As another example, a low frequency can be less than 15 Hz. As a further example, a low frequency can be less than 10 Hz. As another example, a low frequency can be less than 5 Hz.

At Step 44, hyper-excitability of a target neural tissue (e.g., within the cortex of a patient's brain, a hypothalamus of a patient's brain, or the like) that is activated by the corpus callosum can be reduced for the time. The hyper-excitability (which may be indicative of seizure activity or behavior) may be reduced for longer than the time the electrical stimulation signal is applied. The suppression of the seizures (and/or corresponding reduction in hyper-activity) can occur (1) as the stimulation is delivered and (2) may extend for a period after the time (after the stimulation of the corpus callosum is complete). For example, the period after the time can extend for at least two hours longer than the time. As another example, the period after the time can extend for at least four hours longer than the time. As a further example, the period after the time can extend for at least one day longer than the time. As yet another example, the period after the time can extend for at least seven days longer than the time.

FIG. 5 shows a method 50 for configuring an electrical signal for stimulation of the corpus callosum based on feedback control. At 52, an electrical stimulation signal can be applied to the corpus callosum in a patient's brain. The electrical stimulation signal can have one or more configurable properties (such as a type of waveform, a pulse width, a current intensity, a current amplitude, and a voltage amplitude, or the like). At 54, a property of the cortex in the patient's brain can be measured. The property of the cortex can be related to hyper-excitability of the target neural tissue within the cortex. At 56, a configurable parameter of the electrical stimulation signal can be updated based on the measured property of the cortex. The method can then revert back to 52 and the electrical stimulation signal can be updated with the new parameters based on the feedback related to the property of the cortex.

In other instances, the recording electrode can be placed within a focal area to make sure the electrode (e.g., (NE(S)) 14) is placed in the correct location to stimulate the correct axons. An output can be provided (e.g., by feedback unit 16) related to whether the electrode (e.g., (NE(S)) 14) is in the correct place (and/or whether the correct axons are activated in the corpus callosum) and/or can provide an autonomous movement of the electrode (NE(S)) 14 into a more proper position. The output can be provided after the corpus callosum is stimulated.

### V. EXAMPLES

The following examples demonstrate the efficacy of corpus callosum stimulation at low frequencies to suppress cortical seizures. The following examples are for the purpose of illustration only is not intended to limit the scope of the appended claims.

### Example 1

This example demonstrates how low-frequency fiber-tract stimulation of the corpus callosum suppresses both cortical and cortically induced hippocampal seizures.

### Materials and Methods

Animals

All animal procedures were conducted in accordance with the guidelines reviewed and approved by the institutional animal care and use committee of Case Western Reserve University. Twenty-nine adult male Sprague-Dawley rats (150-300 g; Charles River) were used in this study. Isoflurane anesthesia was administered at a concentration of 1%-3%, and vitals were monitored during the entire experiment. Animals were secured via a stereotactic apparatus. Subsequently, a small incision was made along the rostrocaudal axis to expose the skull. All connective tissue and vasculature were removed from the skull by scrubbing with hydrogen peroxide and electric cautery. A total of 10 burr holes were drilled into the skull. Stainless steel monopolar electrodes were placed in the primary motor cortex, in the CA3 region of the hippocampus, and in the corpus callosum at six separate locations along the rostrocaudal axis (diameter=200 μm; Plastics One; part # E363/3). All coordinates were determined via rat brain atlas (motor cortex: anteroposterior [AP]=−1.00 mm, lateral=−2.40 mm, depth=−1.60 mm; hippocampus: AP=−3.14 mm, lateral=−3.00 mm, depth=−3.75 mm; corpus callosum: AP=+0.20, −1.88, −4.16 mm, lateral=±1.00 mm, depth=−3.00, −3.10, −2.60 mm). A stainless steel screw electrode was embedded into the skull near the most posterior bone sutures and used as a reference. All electrodes were secured to the skull using dental cement (TEETS Denture Material; Co-Oral-Ite Dental Manufacturing Company). A 5-μL micro syringe (Hamilton, Reno, Nev.) was secured to the stereotaxic frame and inserted into the contralateral motor cortex (AP=+1.00 mm, lateral=−2.40 mm, depth=−1.60 mm). The location of the electrodes and injection site as well as the corresponding coronal slices of the rodent brain are shown in FIG. 5, element a.

Focal Cortical Model

To test the hypothesis that selective corpus callosum stimulation can reduce seizures, a stable model was developed of focal cortical seizures using repeated local injections of 4-aminopyridine (4-AP). Injections of a cocktail of 30 mmol/L 4-AP, 1.2 mmol/L CaCl2), and 0.6 moll/L MgSO4 were injected at a rate of one 1-μL bolus per hour under a slightly reduced isoflurane anesthesia concentration (<2%). Injections were delivered via a 5-μL syringe as a 1-μL bolus given at the start of every hour during the experiment. The injections were administered to the contralateral cortex to allow seizures to spread through the corpus callosum to the unaffected hemisphere, where was placed recording electrodes in the corresponding motor cortex and hippocampus. The first seizure usually occurred within 20 minutes to 1 hour following the initial injection. To ensure the seizures did not disappear over time, 3 hours of baseline activity were recorded prior to every experiment. The seizure rate was found to actually increase over time. Moreover, it did not take very long for activity to generalize from the motor cortex to the hippocampus, as can be seen in FIG. 5, element b. The total duration of time spent seizing in each of the 3 hours of baseline was almost identical between the cortex and hippocampus.

Data Acquisition and Seizure Identification

Electroencephalographic recordings were sampled at 100 kHz (PowerLab DAQ; AD Instruments) and amplified by 100. Recordings were taken continuously over the course of the experiment to monitor seizures in the cortex and secondarily in the hippocampus. Seizures were identified according to a metric, wherein the electroencephalographic segment must be double the amplitude of the baseline amplitude, the majority of the frequency content must be at least 5 Hz, and this activity must continue for >5 seconds. Seizure onset was identified as the time at which the first spike from the first segment of electroencephalographic activity satisfying these criteria occurred. The duration of each of these seizures was recorded for purposes of comparison across experimental periods. Periods of time during stimulation were cleaned of stimulation artifacts by applying template subtraction and a median filter. The artifact removal did not affect seizure identification.

Electrical Stimulation

Each pair of stimulation electrodes in the corpus callosum was utilized independently of the rest. The appropriate stimulation amplitude was determined by finding 50% of the maximum amplitude of the evoked potential in the cortex using the pair of electrodes closest to the recording electrodes. Based on the evoked potentials in the cortex, the amplitude was fixed at 4 mA for all experiments. Stimulation was delivered through a digital stimulator with a current isolator (DS8000 Digital Stimulator; World Precision Instruments, Sarasota, Fla.) driven by a separate function generator (FG-8002, Goldstar) in the form of a 2-mA biphasic (4 mA peak-peak) current pulse with a 100 microsecond pulse width (each phase was 100 microseconds, giving a total biphasic pulse width of 200 microseconds). The current pulses were delivered continuously for 1 hour at a frequency of 1, 10, 20, or 30 Hz depending on the experiment. The different frequencies were applied only to the pair of electrodes closest to the recording electrodes. Only u20-Hz stimulation was used in the other two pairs of electrodes.

Experimental Design and Statistical Methods

In each experiment, activity was recorded for 3 hours prior to the initiation of any stimulation protocol to validate our acute model. In 14 animals, 20-Hz stimulation was applied to the electrode pair in the corpus callosum positioned closest to the recording electrode in the cortex. Following 1 hour of this stimulation, a period of 1 hour of post stimulation was recorded to monitor for any aftereffect. In sets of five animals, stimulation was tested at 1, 10, or 30 Hz from the same location to compare efficacy. The efficacy of stimulation was tested from the other two pairs of electrodes in the corpus callosum farther from the motor cortex. The experiments conducted using these other two pairs of electrodes were carried out using an additional set of five animals. Finally, the effect of moving the seizure focus was tested, recording, and stimulating electrodes on the efficacy of stimulation. The entire cortical/callosal assembly placement was shifted to a more posterior aspect of the corpus callosal axis. The experiments conducted using this alternate seizure focus location required the use of five additional animals. To compare the efficacy of stimulation at different frequencies and in different locations, a Friedman test was used with Dunn multiple comparisons post hoc test with a significance level of 0.05. A nonparametric analysis of variance was utilized due to the non-normal distribution of these data as determined by a D'Agostino-Pearson omnibus normality test (P<0.05). Total time spent seizing during baseline, stimulation, and post stimulation was compared for statistical significance.

### Results

Seizure Activity During Baseline

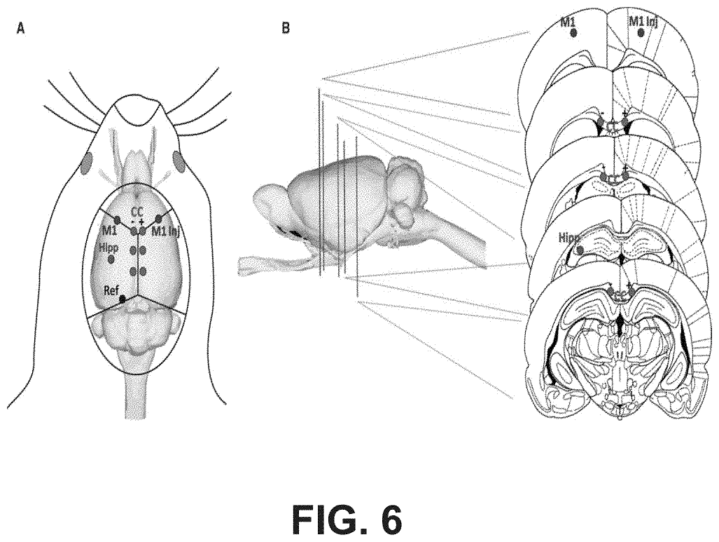

The temporal distribution of seizures generated by this focal model of cortical epileptiform activity was first examined. Two typical examples of seizures recorded are shown in FIG. 6, element a. The seizures generated in the cortex have patterns similar to those observed with hippocampal applications of 4-AP. The seizures typically begin with a single high-amplitude spike and then develop into high-frequency firing followed by lower-frequency high amplitude spikes. The spectrogram in FIG. 6, element a demonstrates the clear difference in power spectral density between seizure activity and the underlying baseline. Seizure activity similar to that observed in the cortex was also generated in the hippocampus, often with an onset delay of several seconds. To determine the stability of seizure activity, the total time spent seizing was measured 3 hours prior to stimulation to establish a baseline. FIG. 6, element b shows that the seizure activity generated by the 4-AP focal model did not decrease over time but increased. The total time spent seizing significantly increased following a 3-hour baseline period compared to the initial seizure activity during the first hour (P<0.0001, n=24). Furthermore, by the end of hour 3, seizures had almost completely generalized to the hippocampus, with the majority of cortical seizures triggering hippocampal seizures. During the first hour of the baseline period, all seizures were <200 seconds long, with a significantly higher proportion of the seizure duration generated in the cortex, as shown in FIG. 6, element c. In the second hour of baseline recording, seizure durations in the hippocampus and cortex were similar, with some seizures lasting as long as 500 seconds (FIG. 6, element d). By the third hour of baseline, the hippocampus and cortex had become nearly synchronized, with individual seizures of the same duration. Moreover, some seizures in both the cortex and hippocampus were observed to last for as long as 10 minutes. The 4-AP model of acute focal cortical seizures produces a stable baseline seizure frequency and duration. This model is also shown to exhibit generalization of seizures in the cortex to the hippocampus. The seizure focus is known to be extensively innervated by corpus callosum fibers and should therefore serve as a suitable model to evaluate the effect of LFS for cortical seizure suppression.

Twenty-Hertz Stimulation Suppresses Cortical Seizures

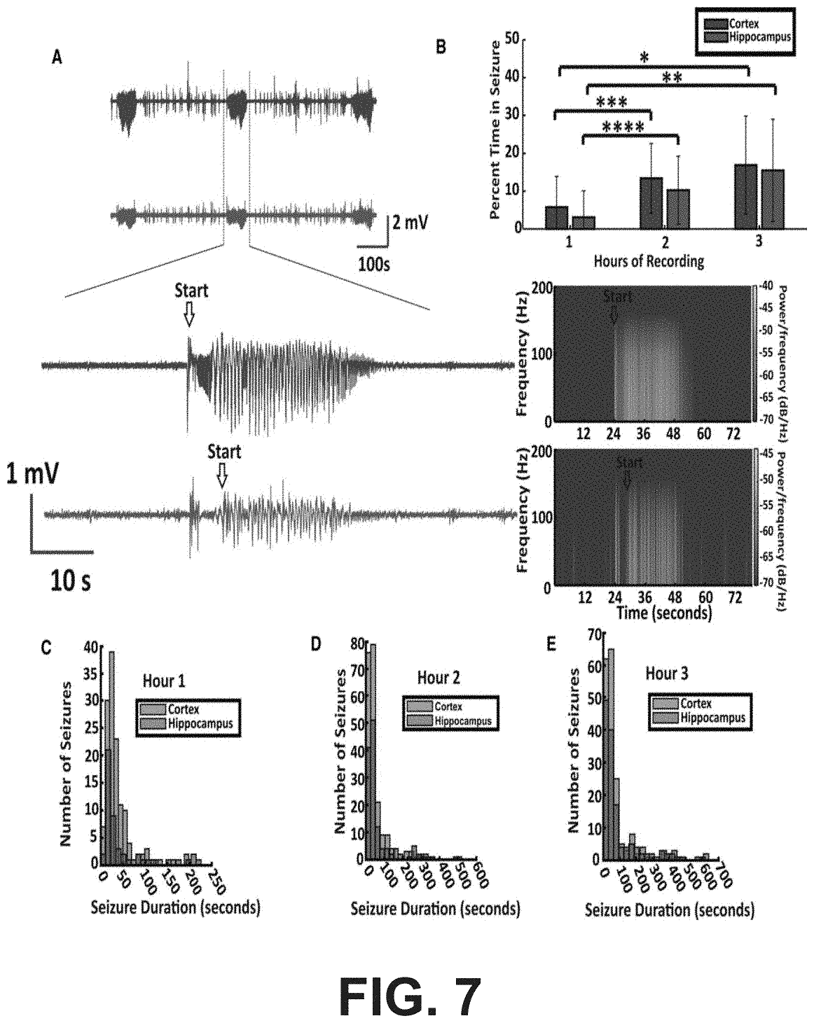

To determine the effect of corpus callosum stimulation on the seizure activity described above, two stimulation electrodes were positioned along the anterior-posterior axis of the corpus callosum to activate specifically those fibers innervating the location of the focus in the cortex. Single pulse stimulation was applied to generate evoked potentials to confirm that the focus was innervated by the fibers activated. In each experiment, 3 hours of baseline activity was recorded followed by a recording period of 1 hour during which stimulation was applied (FIG. 7, element a). FIG. 7, element b shows distinct seizures along with a large number of interictal spikes during 1-Hz stimulation. Similarly, seizures and interictal spiking were prevalent during 30-Hz stimulation. In FIG. 7, element c, only sporadic interictal spiking is observed during 20-Hz stimulation. FIG. 7, element D shows the effect of stimulation at 1, 10, 20, and 30 Hz. Ten and 20 Hz generated a 76% and 95% reduction in seizures, respectively (P<0.05, n=5; P<0.0001, n=14). This effect was most pronounced at 20 Hz, with complete seizure suppression in the majority of experiments. There was no statistically significant effect of either 1 or 30 Hz at suppressing seizures.

Twenty-Hertz Stimulation Also Suppresses Hippocampal Seizures Induced by a Cortical Focus

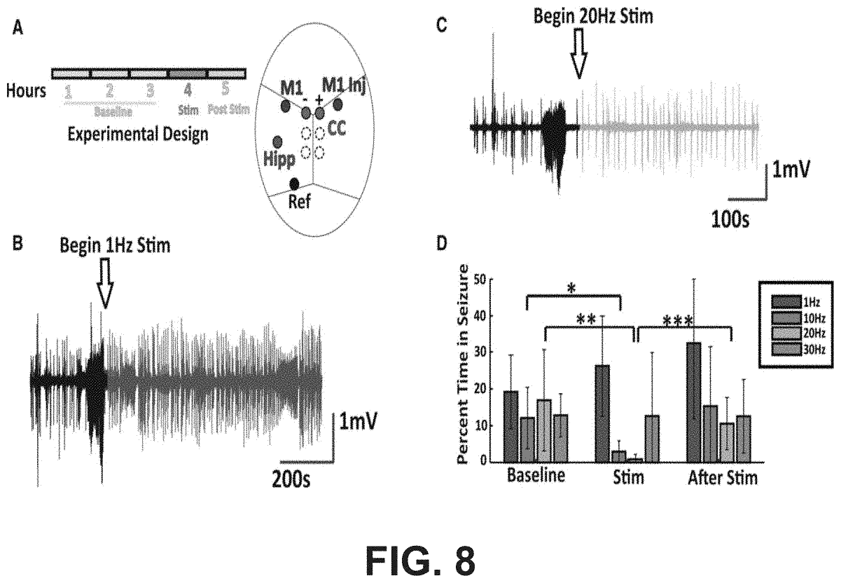

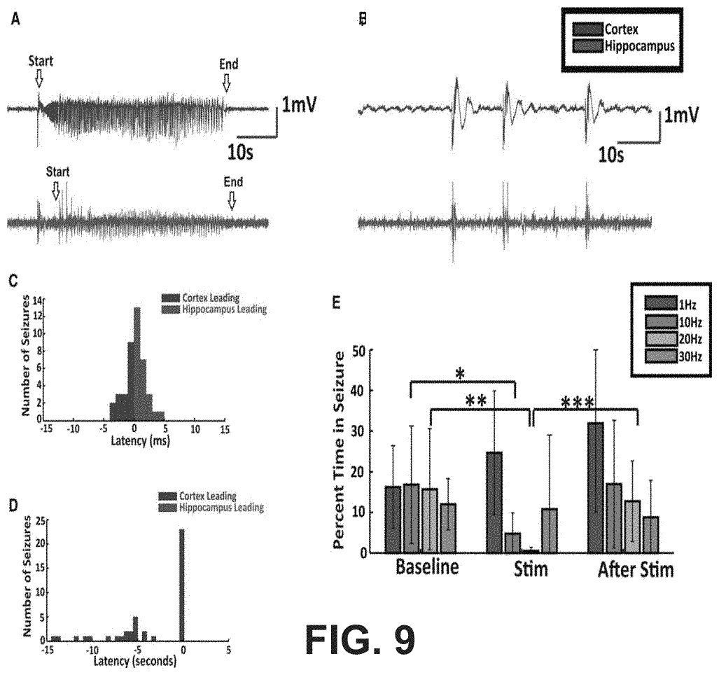

Recordings in the hippocampus show that the cortical seizures generalized to the hippocampus (see FIG. 8, element a). When 20-Hz stimulation was applied to the corpus callosum (FIG. 8, element b), activity in both the cortex and the hippocampus was limited to occasional interictal spiking. The total time spent seizing in the hippocampus was at a minimum during stimulation at 10 and 20 Hz, as was the case in the cortex. It is important to distinguish propagation of individual spikes originating in the cortex from the seizure wavefront itself. In FIG. 8, element c, one can see that the seizure spikes are fairly well synchronized between the hippocampus and cortex, with roughly equal proportions emanating from either structure. The seizure wavefront, however, which was define as the onset of the seizure, always occurs first in the cortex, indicating that there is not an independent seizure focus in the hippocampus, as can be seen in FIG. 8, element d. According to our criteria that seizures must consist of >5-Hz spectral power for >5 seconds, FIG. 2, element A shows the delay between the cortex and hippocampus in terms of both the spectral power and amplitude (>2×baseline amplitude). FIG. 8, element e, shows that 10 and 20 Hz are the only frequencies effective at suppressing seizures, with 20 Hz providing the greatest suppressive effect in the hippocampus (P<0.05, n=5; P=0.0003, n=14).

Stimulation Efficacy is Spatially Selective

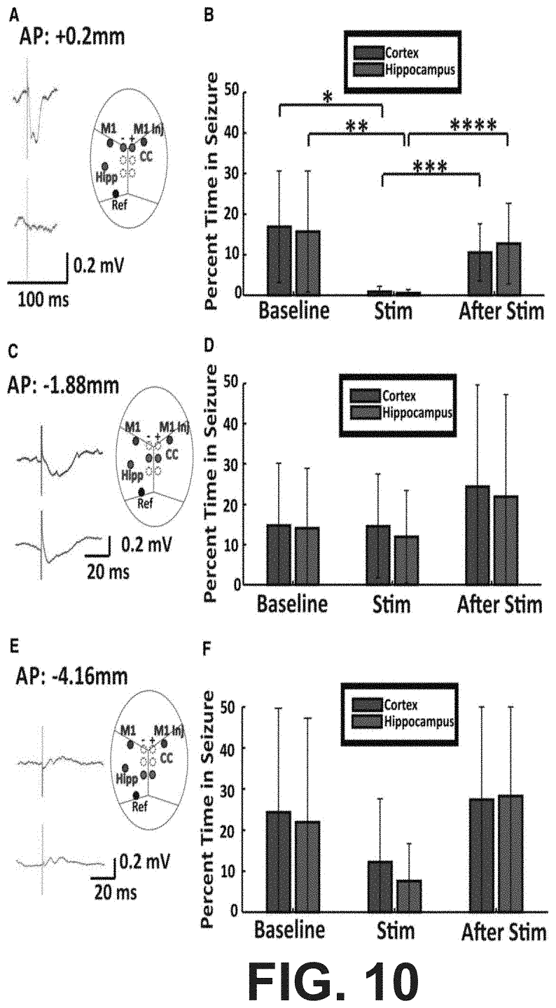

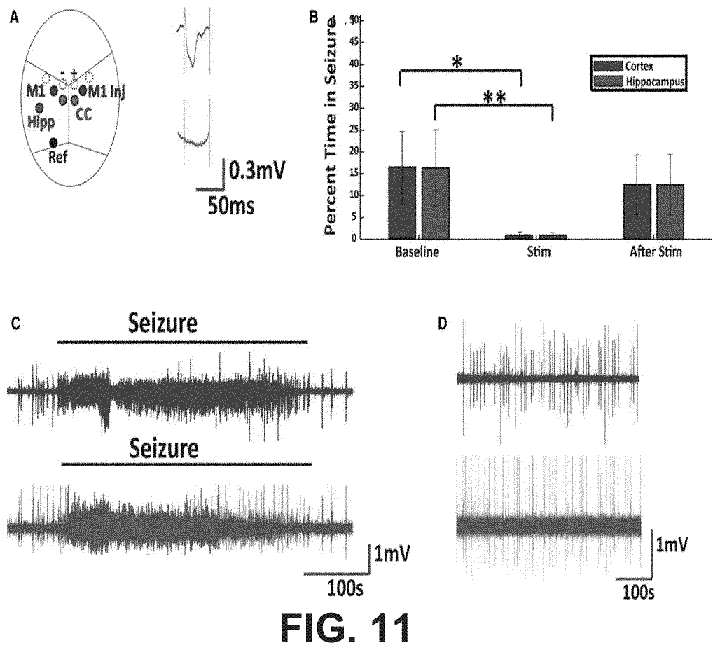

Corpus callosum fibers are topographically organized, and only a subset of those fibers can be activated by a single bipolar electrode pair. The feasibility of selectively activating a cortical region of interest (seizure focus) was determined with corpus callosum stimulation, thereby minimizing the amount of cortical tissue activated and limiting the potential side effects. To determine the spatial selectivity of corpus callosum stimulation, bipolar electrodes were placed at locations along the anterior/posterior axis at +0.2, −1.88, and −4.16 mm (FIG. 10, elements a, c, and e). The stimulation frequency was fixed at 20 Hz based upon our previous findings. In FIG. 10, elements a, c, and e, the evoked potentials from all three stimulation locations are shown. Seizure suppression generated by stimulation electrodes from each location is also shown in FIG. 5, elements b, d, and f, showing that suppression is greatest for electrode positions generating the largest evoked potential. This result indicates that the stimulation paradigm is selective, as even the most proximal stimulation site to the optimal target (2 mm posterior to the optimal portion of the corpus callosum for this particular focus) has no statistically significant effect on seizure suppression (FIG. 10, element d). The stimulation location 4 mm posterior to the optimal portion of the corpus callosum tract also showed no effect on seizure suppression (FIG. 10, element e). To further validate the ability to target specific tracts of the corpus callosum based upon the location of the seizure focus, the seizure focus/recording electrode and stimulating electrodes were moved 2 mm posterior from their original location, as shown in FIG. 11, element a. Similar to the results shown in FIG. 10, seizures were suppressed by 95% during stimulation at the more posterior location (P=0.03, n=5), as shown in FIG. 11, element b. Seizures were also inhibited identically in the hippocampus (P=0.03, n=5).

### Example 2

This example demonstrates that low frequency stimulation of the corpus callosum is the only method of stimulation to significantly reduce seizure frequency.

### Materials and Methods

Surgical Procedure

All animal procedures were conducted in accordance with the guidelines reviewed and approved by the institutional animal care and use committee of Case Western Reserve University. Forty-eight adult male Sprague-Dawley rats (150-300 g; Charles River) were used in this study. Isoflurane was administered at a concentration of between 1-3% and vitals were monitored while the animals were under anesthesia. The rat's head was secured using a stereotactic frame prior to any further manipulation. A small incision was made along the rostrocaudal axis to expose the skull and all connective tissue was removed with manual abrasion with hydrogen peroxide. Subsequently, several burr holes were drilled into the skull for either electrode placement, micro syringe insertion, or transection. Depending on the particular experiment there were different numbers of holes created in the skull to accommodate the stimulation electrodes or transection. In all animals two burr holes were made in the skull over right somatosensory cortex (S1), one was made over the left S1, and another in the most posterior bone sutures of the skull. All coordinates were determined through the use of a rat brain atlas. One electrode was placed in one of the 2 holes over the right S1 (anteroposterior [AP]=−0.4 mm, lateral=2.3 mm, depth=−1.4 mm) another electrode was placed in the contralateral S1 (AP=−1.6 mm, lateral=−2.0 mm, depth=−1.4 mm). A stainless steel screw electrode was placed in the posterior bone sutures and used as a reference for recording. A micro syringe was inserted into the second burr hole over the right S1 (AP=−1.6 mm, lateral=2.0 mm, depth=−1.4 mm). For corpus callosum stimulation, burr holes were drilled in the midline and electrodes were placed in the two holes (AP=−1.3 mm, lateral=+/−0.6 mm, depth−3.0 mm). For focal stimulation an additional hole was made over the right S1 along with a hole over the right posterior bone suture for a screw electrode to be used as a return for stimulation current. The stimulation electrode was placed in the third burr hole over the right S1 (AP=−1.1 mm, lateral=2.2 mm, depth=−1.4 mm). For stimulation of the ANT a screw electrode was also placed in a burr hole over the right posterior bone suture. Also, a burr hole was drilled over the right ANT and an electrode was inserted (AP=−1.5 mm, lateral=1.5 mm, depth=−5.3 mm). To transect a portion of the corpus callosum, 3 large adjacent burr holes were drilled along the midline of the rostro caudal axis of the skull to form a continuous opening for the insertion of a knife (AP=0 to −3 mm, lateral=0 mm). All electrodes were fixed to the surface of the skull with dental cement.

Focal Cortical Model

The procedure used a model similar to what was reported. Briefly, 1 μL injections of a cocktail of 4-aminopyridine (4-AP) were administered once per hour at the beginning of each of the three hours of the experiment in order to maintain spontaneous seizures. The injections were made in the right S1 region to create a seizure focus in the right somatosensory cortex. Anesthetic depth was decreased by lowering the concentration of Isoflurane to <2% to limit the effect of anesthesia on neural excitability.

Data Acquisition and Seizure Identification

Local field potential (LFP) recordings were sampled at 40 kHz and amplified by 100. LFPs were monitored during the entirety of every experiment to determine seizure frequency in the electroencephalogram (EEG). Seizures were identified using the criteria, wherein an EEG segment must have an amplitude greater than 2 times the baseline, the majority of the spectral power must be >5 Hz, and this segment must last for longer than 5 seconds. All EEG segments meeting these criteria were classified as seizures. The duration of these seizures was recorded for purposes of comparison between different experimental time periods. EEG recorded during stimulation was cleaned using template subtraction and a median filter to remove the stimulation artifact using the method utilized.

Deep Brain Stimulation

For CC stimulation the amplitude was determined by finding 50% of the maximum evoked potential amplitude in the S1. Based on this, the stimulation for all experiments involving DBS was set to 4 mA. Electrical current was applied in the form of a 2-mA biphasic (4 mA peak-peak) current pulse with a 100-microsecond pulse width (each phase was 100 microseconds, giving a total biphasic pulse width of 200 microseconds). The current pulses were delivered continuously for 1 hour at either a high-frequency (200 Hz) or a low-frequency (20 Hz). For grey matter stimulation a screw electrode over the posterior ipsilateral cortex was used as a return for the current. CC stimulation was applied between a pair of electrodes positioned parallel to the longitudinal axis of the callosal axons.

Transection

A partial transection was made in some animals along a portion of the rostro caudal axis. A small blade was inserted into the most anterior position in the skull opening down to 4 mm beneath the surface. The knife was then moved caudally until it reached the most posterior position in the opening. The knife was then pulled directly up out of the brain to complete the transection of a 3 mm section of the corpus callosum. Only the region of the corpus callosum innervating the focal region was cut (as determined by evoked potentials).

Experimental Design

Experiments were carried out in 48 male Sprague Dawley rats. In every experiment there was recorded one hour of baseline activity followed by one hour during which either stimulation was applied, a transection was made, or no action was taken (sham condition). Subsequently, observations were recorded for one additional hour to observe any after-effect. For the initial set of experiments 4 groups of 7 animals each (28 total, n=7) were divided into a corpus callosum low-frequency (CC-LFS) group, a focal high-frequency stimulation (Focal-HFS) group, an anterior nucleus of the thalamus high-frequency stimulation (ANT-HFS) group, and a sham group. The CC-LFS group received 1 hour of 4-mA 20 Hz stimulation, both the Focal-HFS and ANT-HFS group received 1 hour of 4-mA 200 Hz stimulation, and the sham group received nothing. In another set of experiments 3 additional groups of animals were added to switch the frequency parameters between white matter and grey matter targets. Each group contained 5 animals and was compared against 5 animals from the previous group of 7 sham animals (15 additional animals, n=5). The animals were split into either the corpus callosum high-frequency (CC-HFS) group, the focal low-frequency (Focal-LFS) group, the anterior nucleus of the thalamus low-frequency (ANT-LFS) group, a corpus callosum group (CC-CUT) or the 5 sham animals from the previous experiments (SHAM). With these experiments the CC group received 1 hour of 4-mA 200 Hz stimulation while both the Focal-LFS and ANT-LFS received 1 hour of 4-mA 20 Hz stimulation. In a final set of experiments a group of 5 animals was subjected to a partial transection (CC-CUT) of the corpus callosum during the second hour of the experiment. For all experiments the total time spent seizing during each hour was normalized to the total time in each period (1 hour). The percent time spent seizing during each hour was compared between each group and the sham group. In order to make these comparisons a Friedman test was used with Dunn multiple comparisons post hoc test with a significance level of 0.05. A nonparametric analysis of variance was utilized due to the non-normal distribution of these data as determined by a D'Agostino-Pearson omnibus normality test (P<0.05).

### Results

Comparison of CC-LFS, Focal-HFS, and ANT-HFS

First, the efficacy of CC-LFS, focal-HFS, and ANT-HFS was compared in suppressing seizures by determining the percent time spent seizing in each group of animals and comparing results to the time-matched sham group. During the first hour of recording seizures began at the site of injection in the S1 after about 10 minutes and spread to the contralateral S1 after another 5 to 10 minutes. The seizures were typically larger in amplitude in the seizure focus than in the mirror focus. Seizures demonstrated the same characteristics with high-frequency and amplitude segments that follow discrete patterns and repeat frequently.

The corpus callosum low-frequency stimulation group was the only stimulation group that experienced a reduction in seizures. During CC-LFS, activity consisted of limited to short bursts of spikes (<5 seconds) in the mirror focus and occasional brief seizures in the cortical focus itself. CC-LFS reduced seizures by 65% (p=0.0014, n=7) in the seizure focus and by 97% (p=0.0026, n=7) in the contralateral mirror focus. There were no significant differences between the other stimulation techniques and the sham group. Although non-significant, focal-HFS and ANT-HFS generated an increase instead of an expected decrease in seizure duration. Recordings in the focal region showed that focal-HFS increased the percent change in time spent seizing during stimulation by 6.6%+/−17% while ANT-HFS generated a 9.1%+/−18% increase. In the mirror focal-HFS and ANT-HFS produced a 25%+/−19% and 18%+/−22% increase in time spent seizing respectively.

Effect of Location Vs. Frequency on Efficacy

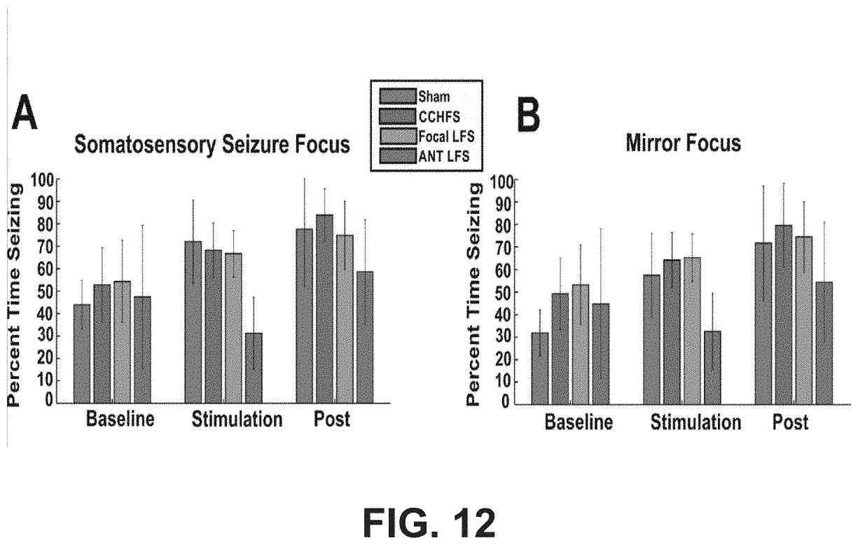

To determine if this disparity in effect might be due to a difference in only one of the parameters rather than the combination of both location and frequency, the parameter pairings were reversed in several additional groups of animals. High-frequency stimulation was applied to the corpus callosum and low-frequency stimulation to the seizure focus and anterior nucleus of the thalamus. Surprisingly, none of these pairings resulted in a decrease in seizures in either the seizure focus (FIG. 12, element A) or the mirror focus (FIG. 12, element B).

During focal-LFS there was a non-significant 7.4%+/−15% decrease and 14%+/−15% increase in seizure duration in the focus and mirror focus respectively. When LFS was applied to the ANT a non-significant 57%+1-17% and a 43%+1-18% decrease in (I percent time spent seizing in the focus and mirror focus respectively were observed. Neither reduction was statistically significant although the effect in the focus was very close to the 5% significance threshold (p=0.0592, n=5). Applying HFS to the corpus callosum resulted in a non-significant 5%+/−16% reduction and a 12%+/−16% increase in seizure duration in the focus and mirror focus respectively.

Comparison Between CC Transection and CC-LFS

Instead of applying electrical stimulation a transection of the corpus callosum was made within the region responsible for reciprocally innervating the seizure focus and mirror focus. Transecting this region of the corpus callosum allowed for direct comparison of the efficacy of a corpus callosotomy to CC-LFS in seizure suppression.

Following a one-hour baseline period, a blade was lowered from the surface of the brain to a region below the CC and moved along the anteroposterior axis to cut only those fibers innervating the focus. Immediately following the transection, activity in both the focus and mirror focus decreased dramatically. However, the seizure activity gradually returned. Typically, after about 40 minutes, seizure activity returned to sham levels. During the first hour following the transection, only the seizure focus showed a 65%+/−18% reduction in seizures (p=0.016, n=5). The reduction in seizures occurring in the seizure focus caused by a CC transection was comparable to CC-LFS with no significant difference between the CC-transection group and the stimulation group. There was a 57%+/−18% non-statistically significant reduction in seizures in the contralateral cortex (p=0.1381, n=5). In comparison, the CC-LFS group demonstrated a seizure suppression of 97% in the contralateral cortex that was significantly more effective than callosotomy of the same fibers used for stimulation and is a reversible procedure.

From the above description, those skilled in the art will perceive improvements, changes and modifications. Such improvements, changes and modifications are within the skill of one in the art and are intended to be covered by the appended claims.

The following is claimed:

1. A method comprising:
applying an electrical stimulation signal with a frequency of less than 50 Hz to a corpus callosum in a patient's brain for a time; and
reducing hyper-excitability of a target neural tissue within a cortex of the patient's brain that is activated by the corpus callosum, wherein the hyper-excitability is reduced for at least the time.

2. The method of claim 1, wherein the hyper-excitability is reduced for at least two hours longer than the time.

3. The method of claim 1, wherein the hyper-excitability is reduced for at least four hours longer than the time.

4. The method of claim 1, wherein the hyper-excitability is reduced for at least one day longer than the time.

5. The method of claim 1, wherein the hyper-excitability is reduced for at least seven days longer than the time.

6. The method of claim 1, wherein the electrical stimulation signal is applied by an implanted stimulating electrode at a position and an orientation within the corpus callosum based on a target focal area.

7. The method of claim 1, wherein the hyper excitability is indicative of seizure behavior.

8. The method of claim 1, wherein the electrical stimulation signal is applied according to one or more configurable properties by an implanted stimulating electrode.

9. The method of claim 8, wherein the one or more configurable properties comprise at least one of a type of waveform, a pulse width, a current intensity, a current amplitude, and a voltage amplitude.

10. The method of claim 9, wherein the one or more configurable parameters is updated based on a feedback signal that indicates the hyper-excitability of the target neural tissue within the cortex of the patient's brain.

11. A system comprising:
a stimulation generator configured to generate an electrical stimulation signal with a frequency of less than 50 Hz; and
at least one electrode configured to apply the electrical stimulation signal to a corpus callosum in a patient's brain for a time; and
wherein hyper-excitability of a target neural tissue within a cortex of the patient's brain that is activated by the corpus callosum is reduced for at least the time.

12. The system of claim 11, wherein the electrode is implanted within the patient's brain at a position and an orientation within the corpus callosum based on a target focal area.

13. The system of claim 12, wherein the position and/or the orientation is altered based on a feedback signal indicative of the reduction in the hyper-excitability.

14. The system of claim 11, wherein at least one of a type of waveform, a pulse width, a current intensity, a current amplitude, and a voltage amplitude are changed in response to a feedback signal.

15. The system of claim 11, wherein the hyper excitability is indicative of a likelihood of incoming seizures.

16. The system of claim 11, wherein the hyper-excitability is reduced for at least two hours longer than the time.

17. The system of claim 11, wherein the hyper-excitability is reduced for at least four hours longer than the time.

18. The system of claim 11, wherein the hyper-excitability is reduced for at least one day longer than the time.

19. The system of claim 11, wherein the hyper-excitability is reduced for at least seven days longer than the time.

20. The system of claim 11, wherein the stimulation generator further comprises a feedback circuit to receive a feedback signal from at least one recording electrode.
